(19) United States
(12) Patent Application Publication
(10) Pub. No.: US 2026/0351411 A1
(43) Pub. Date: Nov. 5, 2026

(54) SYSTEMS AND METHODS FOR EXECUTING A PLURALITY OF MACHINE LEARNING MODELS

(71) Applicant: Medtronic, Inc., Minneapolis, MN (US)

(72) Inventors: Kristin N. Hageman, Dayton, MN (US); Joshua J. Nedrud, Roseville, MN (US); Joel A. Anderson, Brooklyn Park, MN (US); Thaddeus S. Brink, St. Paul, MN (US); Philip E. Tracton, Porter Ranch, CA (US); Malgorzata M. Siorek, Blaine, MN (US)

(21) Appl. No.: 19/659,387

(22) Filed: Apr. 27, 2026

Related U.S. Application Data
(60) Provisional application No. 63/799,058, filed on May 2, 2025.

(51) Int. Cl. G16H 50/20; G06N 20/00; G16H 10/60; G16H 40/63

(52) U.S. Cl. CPC G16H 50/20; G06N 20/00; G16H 10/60; G16H 40/63

(57) ABSTRACT

An example implantable medical device includes a memory configured to store: first physiological data of a patient, second physiological data of the patient, a first machine learning (ML) model, and a second ML model; a ML model hardware accelerator; and processing circuitry configured to: apply the first physiological data and the first ML model to the ML model hardware accelerator to generate a first output; and apply at least one of the first physiological data or the second physiological data and the second ML model to the ML model hardware accelerator to generate a second output.

### CROSS-REFERENCE TO RELATED APPLICATION

This application claims the benefit of and priority to U.S. Provisional Patent Application No. 63/799,058 filed May 2, 2025, the entire disclosure of which is incorporated by reference herein.

### TECHNICAL FIELD

The disclosure relates generally to medical device systems and, more particularly, medical device systems configured to detect physiological signals.

### BACKGROUND

Medical devices may be used to monitor physiological signals of a patient. For example, some medical devices are configured to sense cardiac electrogram (EGM) signals indicative of the electrical activity of the heart via electrodes, electroencephalogram (EEG) signals indicative of the electrical activity of the heart via electrodes, and/or an evoked compound action potential (ECAP). Some medical devices may be configured to deliver a therapy in conjunction with or separate from the monitoring of physiological signals.

### SUMMARY

A medical device, such as an implantable medical device (IMD), may be configured to sense one or more physiological signals of a patent. Example physiological signals may include at least one electroencephalogram (EEG) signal, electrocardiogram (ECG) signal, respiration, blood pressure, activity level, electromyography (EMG) signal, evoked potential, evoked compound action potential (ECAP), intrinsic nerve activity, and/or electrophysiology (EP). These signals may be sensed using one or more sensors, such as one or more electrodes, accelerometers, or any other sensors.

As described herein, a system may be configured to perform at least one of executing a plurality of machine learning (ML) models using a shared ML model hardware accelerator, running at least a first ML model and a second ML model in parallel, or running at least a first ML model and a second ML model in series to improve efficiency of using computational resources, such as memory or power, and/or improve efficiency of running a plurality of ML models. For example, running a plurality of ML models using a shared ML model hardware accelerator may enable a plurality of ML models to be run on an IMD, which may improve diagnostic and/or therapeutic accuracy of an IMD.

For example, an IMD having a plurality of ML models, in accordance with techniques described herein, that is configured to operate with improved computational efficiency may enable the IMD to have a greater lifespan and/or enable the IMD to run the plurality of ML models more often, which may help produce improved clinical results for a patient.

In one example, this disclosure describes an implantable medical device comprising: a memory configured to store: first physiological data of a patient, second physiological data of the patient, a first machine learning (ML) model, and a second ML model; a ML model hardware accelerator; and processing circuitry configured to: apply the first physiological data and the first ML model to the ML model hardware accelerator to generate a first output; and apply at least one of the first physiological data or the second physiological data and the second ML model to the ML model hardware accelerator to generate a second output.

In another example, this disclosure describes a method comprising: sensing, by an implantable medical device (IMD), first physiological data of a patient; sensing, by the IMD, second physiological data of the patient; applying, by processing circuitry of the IMD, the first physiological data and a first machine learning (ML) model embedded on the IMD to a ML model hardware accelerator to generate a first output; and applying, by the processing circuitry, at least one of the first physiological data or the second physiological data and a second ML embedded on the IMD model to the ML model hardware accelerator to generate a second output.

The summary is intended to provide an overview of the subject matter described in this disclosure. It is not intended to provide an exclusive or exhaustive explanation of the systems, device, and methods described in detail within the accompanying drawings and description below. Further details of one or more examples of this disclosure are set forth in the accompanying drawings and in the description below. Other features, objects, and advantages will be apparent from the description and drawings, and from the claims.

### BRIEF DESCRIPTION OF THE DRAWINGS

FIG. 1A illustrates the environment of an example medical system in conjunction with a patient, in accordance with one or more techniques disclosed herein.

FIG. 1B illustrates the environment of an example medical system in conjunction with a patient, in accordance with one or more techniques disclosed herein.

FIG. 2 is a functional block diagram illustrating an example configuration of the implantable medical device (IMD) of the medical system of FIGS. 1A or 1B in accordance with one or more techniques disclosed herein.

FIGS. 3A-3B are conceptual diagrams illustrating example techniques of executing a plurality of ML models using a shared ML model hardware accelerator, in accordance with one or more techniques disclosed herein.

FIG. 4A is a conceptual diagram illustrating example processes for running a first ML model and a second ML model in parallel, in accordance with one or more techniques disclosed herein.

FIG. 4B is a conceptual diagram illustrating example processes for running a first ML model and a second ML model in series, in accordance with one or more techniques disclosed herein.

FIG. 5 is a functional block diagram illustrating an example configuration of the external device of FIGS. 1A-1B, in accordance with one or more techniques disclosed herein.

FIG. 6 is a block diagram illustrating an example system that includes an access point, a network, external computing devices, such as a server, and one or more other computing devices, which may be coupled to the IMD and external device of FIGS. 1–5, in accordance with one or more techniques disclosed herein.

FIG. 7 is a flow diagram illustrating an example technique for executing a plurality of ML models using a shared ML model hardware accelerator, in accordance with one or more techniques disclosed herein.

FIG. 8 is a flow diagram illustrating an example technique for running a first ML model and a second ML model in parallel, in accordance with one or more techniques disclosed herein.

FIG. 9 is a flow diagram illustrating an example technique for running a first ML model and a second ML model in series, in accordance with one or more techniques disclosed herein.

Like reference characters denote like elements throughout the description and figures.

### DETAILED DESCRIPTION

A variety of types of implantable medical devices (IMDs) sense physiological signals that include physiological data, such as cardiac EGMs, EEGs, or ECAPs. In some examples, EGMs may also include electrocardiogram (ECGs or EKGs). In some examples, electrodes used by IMDs to sense physiological signals are typically integrated with a housing of the IMD and/or coupled to the IMD via one or more elongated leads. Example IMDs that monitor physiological data and/or provide therapy include spinal cord stimulators, deep brain stimulation devices, and pacemakers and implantable cardioverter-defibrillators. These devices may be coupled to intravascular and/or extravascular leads, as well as include with housings configured for implantation within the heart or other anatomical location that are leadless (e.g., carry electrodes on the housing). An example of pacemaker configured for intracardiac implantation is the Micra™ Transcatheter Pacing System, available from Medtronic plc. Some IMDs that do not provide therapy, e.g., implantable patient monitors, sense cardiac EGMs. Examples of such an IMD are the Reveal LINQ™ and LINQ II™ Insertable Cardiac Monitors, available from Medtronic plc, which may be inserted subcutaneously. Such IMDs may facilitate relatively longer-term monitoring of patients during normal daily activities and may periodically transmit collected data to a network service, such as the Medtronic Carelink™ Network.

In some examples, an IMD attempting to sense a particular signal may also sense noise, and on some occasions the amount of noise included in the sensed signal may make the desired sensed signal, such as an ECAP, EEG signal, or ECG signal, more difficult to identify in a signal sensed by the IMD. In some examples, an IMD may input one or more of the sensed parameters or features derived from the sensed parameters into one or more ML models to detect a presence or features of a particular signal, such as an ECAP, EEG signal, or ECG signal, in the sensed data. In some examples, an IMD may input one or more of the sensed physiological data or features derived from the sensed physiological data into one or more ML models to determine one or more physiological parameters, diagnostic parameters, and/or therapeutic parameters.

An IMD may have limited battery and/or limited memory size for an IMD to be small enough to be effectively implantable in a patient. The power and/or memory needs for running ML models are often high, which may be problematic for an IMD to run a plurality of ML models due to the limited battery and/or the limited memory size of an IMD. When a ML model is operating on an IMD, computational resources and/or power may be limited due to size limitations of an IMD.

Any medical device configured to sense physiological data via implanted electrodes or sensors, including the examples identified herein, may implement the techniques of this disclosure for executing a plurality of ML models using a shared ML model hardware accelerator, for running a first ML model and a second ML model in parallel, and/or for running a first ML model and a second ML model in series.

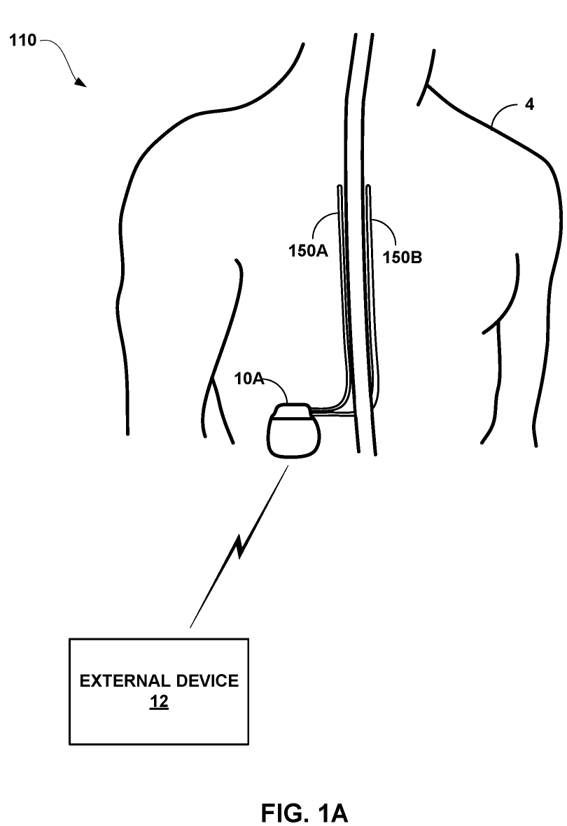

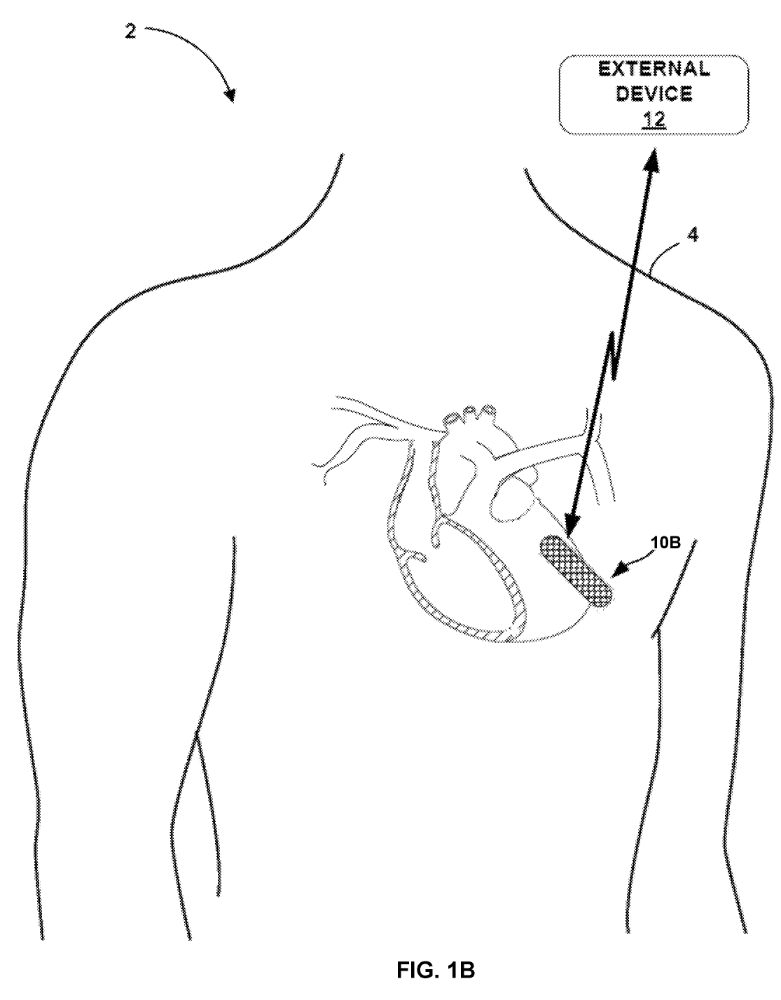

FIG. 1A illustrates a conceptual diagram illustrating an example system 110 that includes another example of an IMD 10A that is configured to deliver spinal cord stimulation (SCS) therapy and an external device 12, in accordance with one or more techniques of this disclosure.

Although the techniques described in this disclosure are generally applicable to a variety of IMDs, application of such techniques to IMDs and, more particularly, an ICM or implantable electrical stimulators (e.g., neurostimulators) will be described for purposes of illustration. More particularly, the disclosure will refer to an IMD 10A system or implantable SCS system for purposes of illustration, but without limitation as to other types of medical devices or other therapeutic applications of medical devices.

As shown in FIG. 1A, system 110 includes an IMD 10A, leads 150A and 150B, and external programmer 150 shown in conjunction with a patient 4, who is ordinarily a human patient. In the example of FIG. 1, IMD 10A is an implantable electrical stimulator that is configured to generate and deliver electrical stimulation therapy to patient 4 via one or more electrodes of electrodes of leads 150A and/or 150B (collectively, “leads 150”), e.g., for relief of chronic pain or other symptoms. In other examples, IMD 10A may be coupled to a single lead carrying multiple electrodes or more than two leads each carrying multiple electrodes. In some examples, the stimulation signals, or pulses, may be configured to elicit detectable ECAP signals that are sensed by IMD 10A. In some examples, IMD 10A may use sensed ECAP signals to determine a posture state occupied by patient 4 and/or determine how to adjust one or more parameters that define stimulation therapy. In this manner, IMD 10A may sense one or more physiological signals and use one or more characteristics of those signals as feedback for closed-loop control of the electrical stimulation that is delivered. IMD 10A may be a chronic electrical stimulator that remains implanted within patient 4 for weeks, months, or even years. In other examples, IMD 10A may be a temporary, or trial, stimulator used to screen or evaluate the efficacy of electrical stimulation for chronic therapy. In one example, IMD 10A is implanted within patient 4, while in another example, IMD 10A is an external device coupled to percutaneously implanted leads. In some examples, IMD 10A uses one or more leads, while in other examples, IMD 10A is leadless.

FIG. 1B illustrates the environment of another example medical system 2 in conjunction with a patient 4, in accordance with one or more techniques of this disclosure. The example techniques may be used with an IMD 10B, which may be in wireless communication with at least one of external device 12 and other devices not pictured in FIG. 1. In some examples, IMD 10B is implanted outside of a thoracic cavity of patient 4 (e.g., subcutaneously in the pectoral location illustrated in FIG. 1). IMD 10B may be positioned near the sternum near or just below the level of the heart of patient 4, e.g., at least partially within the cardiac silhouette. IMD 10B includes a plurality of electrodes (not shown in FIG. 1) and is configured to sense a cardiac EGM via the plurality of electrodes. In some examples, IMD 10A takes the form of the Reveal LINQ™ or LINQ II™ ICM, or another ICM similar to, e.g., a version or modification of, the Reveal LINQ™ or LINQ II™ ICMs.

External device 12 may be a computing device with a display viewable by the user and an interface for providing input to external device 12 (i.e., a user input mechanism). In some examples, external device 12 may be a notebook computer, tablet computer, workstation, one or more servers, cellular phone, personal digital assistant, or another computing device that may run an application that enables the computing device to interact with IMD 10A or IMD 10B. External device 12 is configured to communicate with IMD 10A or IMD 10B and, optionally, another computing device (not illustrated in FIG. 1), via wireless communication. External device 12, for example, may communicate via near-field communication technologies (e.g., inductive coupling, NFC or other communication technologies operable at ranges less than 10–20 cm) and far-field communication technologies (e.g., RF telemetry according to the 802.11 or Bluetooth® specification sets, or other communication technologies operable at ranges greater than near-field communication technologies).

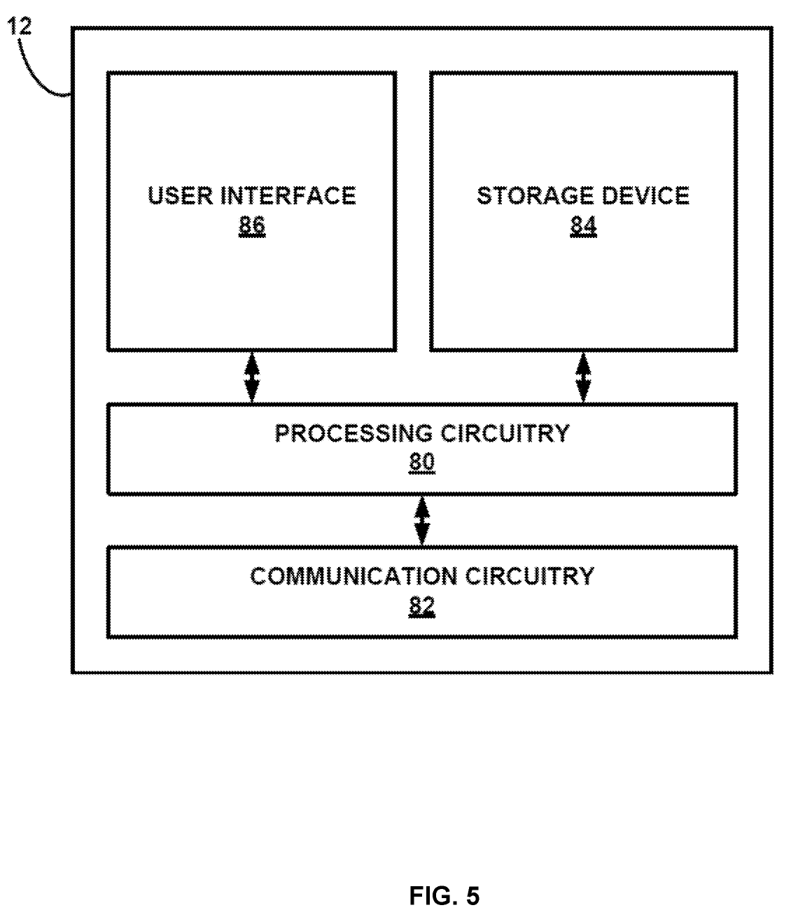

External device 12 may be used to configure operational parameters for IMD 10A or IMD 10B. External device 12 may be used to retrieve data from IMD 10A or IMD 10B. The retrieved data may include values of physiological parameters measured by IMD 10A or IMD 10B, indications of patient health episodes or other maladies detected by IMD 10A or IMD 10B, and physiological signals recorded by IMD 10A or IMD 10B. For example, external device 12 may retrieve physiological data recorded by IMD 10A or IMD 10B. As discussed in greater detail below with respect to FIG. 5, one or more remote computing devices may interact with IMD 10A or 10B in a manner similar to external device 12, e.g., to program IMD 10A or 10B and/or retrieve data from IMD 10A or 10B, via a network.

Processing circuitry of medical system 2, e.g., of IMD 10A, IMD 10B, external device 12, and/or of one or more other computing devices, may be configured to perform the example techniques of this disclosure for determining which sets of layers of a ML model to deactivate or activate to analyze sensed data.

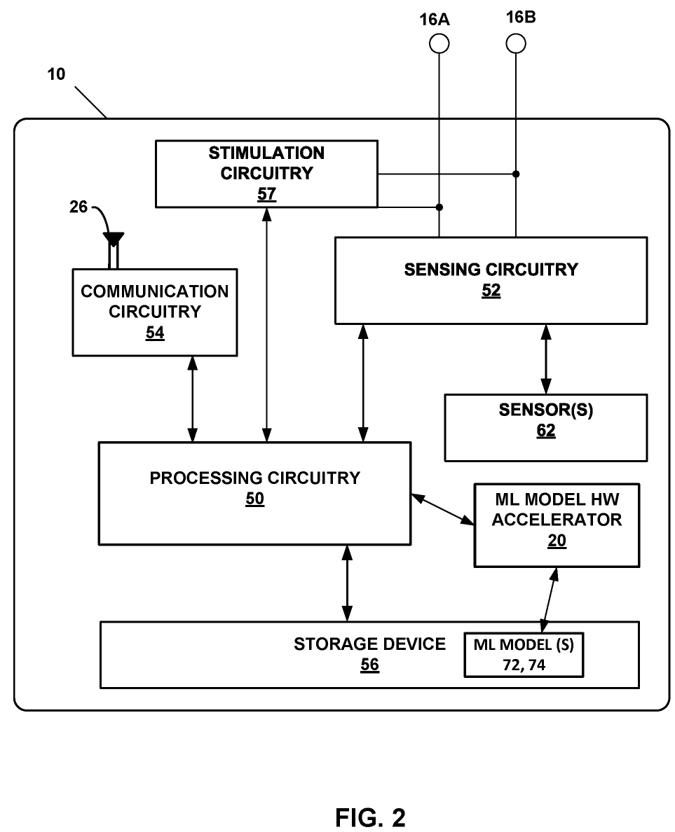

FIG. 2 is a functional block diagram illustrating an example configuration of IMD 10, which is example of IMD 10A of FIG. 1A or IMD 10B of FIG. 1B in accordance with one or more techniques described herein. In the illustrated example, IMD 10 includes electrodes 16A and 16B (collectively “electrodes 16”), antenna 26, ML model hardware accelerator 20, processing circuitry 50, sensing circuitry 52, communication circuitry 54, storage device 56, stimulation circuitry 57, and sensors 62. Although the illustrated example includes two electrodes 16, IMDs including or coupled to more than two electrodes 16 may implement the techniques of this disclosure in some examples. However, IMD 10 does not need to include all these components in some examples, or IMD 10 may include additional components in some examples. For example, IMD 10 may not include stimulation circuitry 57, in some examples.

Processing circuitry 50 may include fixed function circuitry and/or programmable processing circuitry. Processing circuitry 50 may include any one or more of a microprocessor, a controller, a digital signal processor (DSP), an application specific integrated circuit (ASIC), a field-programmable gate array (FPGA), or equivalent discrete or analog logic circuitry. In some examples, processing circuitry 50 may include any volatile, non-volatile, magnetic, optical, or electrical media, such as a random access memory (RAM), read-only memory (ROM), non-volatile RAM (NVRAM), electrically-erasable programmable ROM (EEPROM), flash memory, or any other digital media. In some examples, processing circuitry 50 may include storage device 56. In some examples, processing circuitry 50 may include multiple components, such as any combination of one or more microprocessors, one or more controllers, one or more DSPs, one or more ASICs, or one or more FPGAs, as well as other discrete or integrated logic circuitry. The functions attributed to processing circuitry 50 herein may be embodied as software, firmware, hardware or any combination thereof.

Sensing circuitry 52 may sense signals from electrodes 16 or sensors 62, e.g., to produce a cardiac EGM, EEG signal, ECAP signal, or other signal in order to facilitate monitoring one or more particular physiological signals of the patient. Stimulation circuitry 57 may be coupled to electrodes 16 and configured to cause electrical stimulation therapy to be delivered to patient 4 via one or more electrodes 16. Sensing circuitry 52 also may monitor signals from sensors 62, which may include one or more accelerometers, pressure sensors, and/or optical sensors, as examples. In some examples, sensing circuitry 52 may include one or more filters and amplifiers for filtering and amplifying signals received from electrodes 16 and/or sensors 62.

Sensing circuitry 52 may also provide one or more digitized physiological signals to processing circuitry 50 for analysis, e.g., to apply the physiological signals to sets of layers of ML models 72, 74 embedded on IMD 10 to generate a respective output. In some examples, processing circuitry 50 may store the digitized physiological signals in storage device 56.

In some examples, ML model hardware accelerator 20 may be its own independent circuit in IMD 10. In some examples, ML model hardware accelerator 20 may be co-located with storage device 56. In some examples, ML model hardware accelerator 20 may be positioned in processing circuitry 50 or sensing circuitry 52. In some examples, while FIG. 2 shows ML model hardware accelerator 20 connected to ML model(s) 72, 74, ML model hardware accelerator 20 may additionally or alternatively be connected to storage device 56.

Communication circuitry 54 may include any suitable hardware, firmware, software or any combination thereof for communicating with another device, such as external device 12, another networked computing device, or another IMD or sensor. Under the control of processing circuitry 50, communication circuitry 54 may receive downlink telemetry from, as well as send uplink telemetry to external device 12 or another device with the aid of an internal or external antenna, e.g., antenna 26. In addition, processing circuitry 50 may communicate with a networked computing device via an external device (e.g., external device 12) and a computer network, such as the Medtronic CareLink® Network. Antenna 26 and communication circuitry 54 may be configured to transmit and/or receive signals via inductive coupling, electromagnetic coupling, Near Field Communication (NFC), Radio Frequency (RF) communication, Bluetooth, WiFi, or other proprietary or non-proprietary wireless communication schemes.

In some examples, storage device 56 includes computer-readable instructions that, when executed by processing circuitry 50, cause IMD 10 and processing circuitry 50 to perform various functions attributed to IMD 10 and processing circuitry 50 herein. Storage device 56 may include any volatile, non-volatile, magnetic, optical, or electrical media, such as a random access memory (RAM), read-only memory (ROM), non-volatile RAM (NVRAM), electrically-erasable programmable ROM (EEPROM), flash memory, or any other digital media. Storage device 56 may store one or more ML models 72, 74, such as described herein. Storage device 56 may store, as examples, programmed values for one or more operational parameters of IMD 10 and/or data collected by IMD 10 for transmission to another device using communication circuitry 54. Data stored by storage device 56 and transmitted by communication circuitry 54 to one or more other devices may include digitized cardiac EGMs, as examples.

In some examples, stimulation circuitry 57 is configured to generate and deliver electrical therapy to the patient. Stimulation circuitry 57 may include one or more pulse generators, capacitors, and/or other components capable of generating and/or storing energy to deliver as electrical stimulation therapy.

With respect to the techniques described herein one or more of first ML model 72, 72A, 72B and second ML model 74, 74A, 74B (collectively ML model(s) 72, 74) may be an example of a deep learning model, or deep learning algorithm, trained to generate a particular output. One or more of IMD 10, external device 12, server 94, and/or computing device(s) 100 may train, store, and/or utilize ML model(s) 72, 74, but other devices may apply inputs associated with a particular patient to ML model(s) 72, 74 in other examples. As discussed above, other types of machine learning and deep learning models or algorithms may be utilized in other examples. For examples, a convolutional neural network model of ResNet-18 may be used. Some non-limiting examples of models that may be used for transfer learning include AlexNet, VGGNet, GoogleNet, ResNet50, or DenseNet, etc. Some non-limiting examples of machine learning techniques include Support Vector Machines, K-Nearest Neighbor algorithm, and Multi-layer Perceptron.

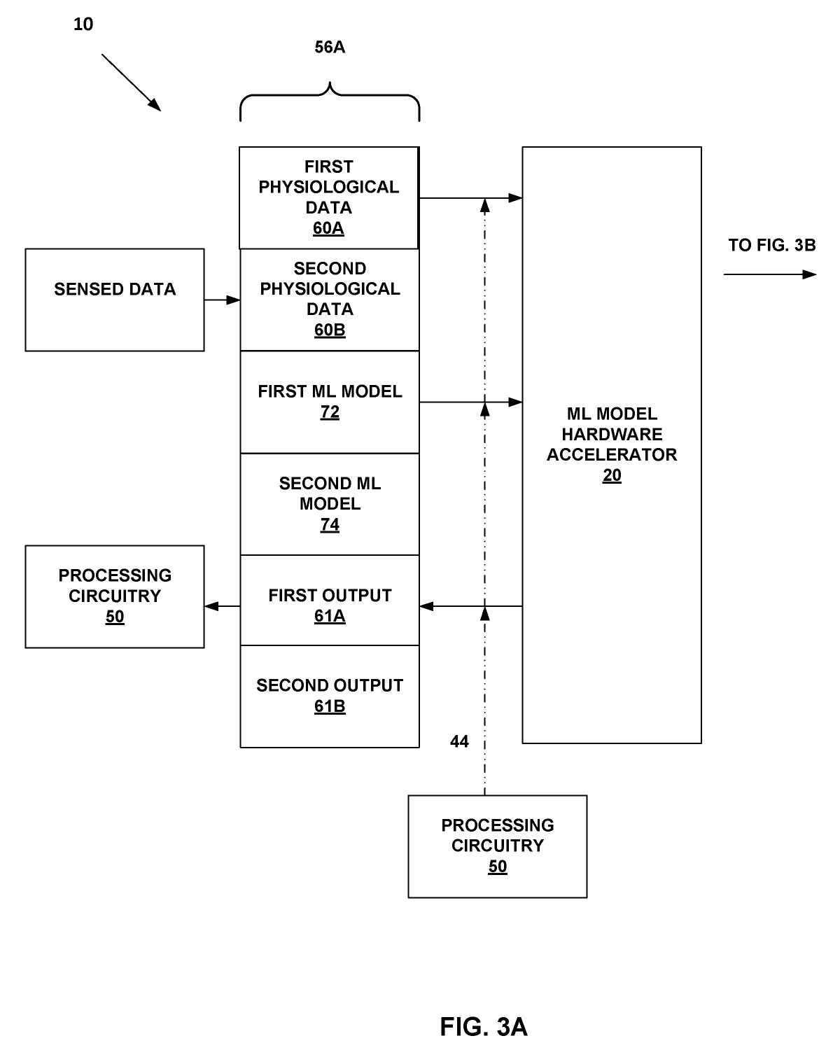

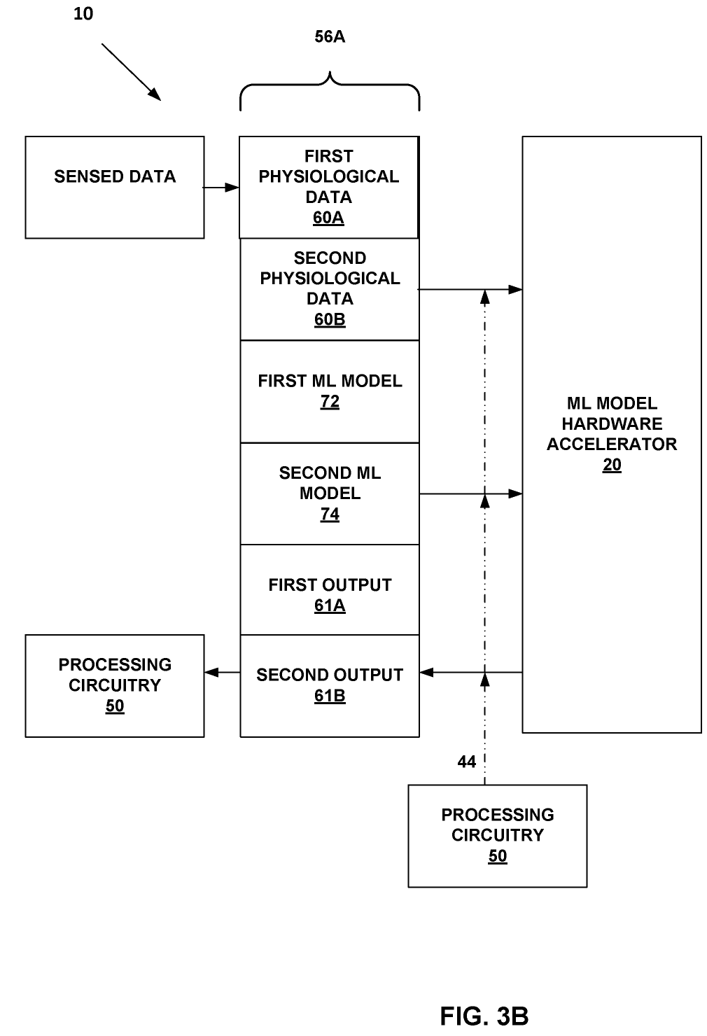

Although the techniques with respect to FIGS. 3A-3B, 4A-4B, and 7-9 are described as being performed by processing circuitry 50 of IMD 10, such techniques may be performed, in whole or part, by processing circuitry of any one or more devices of system 2, such as processing circuitry 80 of external device 12, processing circuitry 98 of server 94, or processing circuitry of one or more computing devices 100.

FIGS. 3A-3B are conceptual diagrams illustrating an example technique of IMD 10 configured to execute a first ML model 72 and a second ML model 74 using a shared ML model hardware accelerator 20. In some examples, IMD 10 may comprise a memory 56A, processing circuitry 50, and a ML model hardware accelerator 20. In some examples, memory 56A may be configured to store first physiological data 60A that is sensed by a first sensor (not shown) of IMD 10 and store second physiological data 60B that is sensed by a second sensor (not shown) of IMD 10. In some examples, the first sensor is configured to sense a different type of signal than the second sensor. In some examples, memory 56A may be configured to store first ML model 72 and second ML model 74.

In some examples, sensing circuitry 52 and/or processing circuitry 50 may input the first physiological data 60A and second physiological data 60B into memory 56A. In some examples, memory 56A may comprise particular sections to respectively store the first physiological data 60A and the second physiological data 60B. For examples, memory 56A may include a first input section to store first physiological data 60A and include a second input section to store second physiological data 60B. In some examples, memory 56A may be a dedicated ML model dedicated memory of storage device 56.

In some examples, first ML model 72 may be trained on data corresponding to the first physiological data 60A and second ML model 74 may be trained on data corresponding to second physiological data 60B. In some examples, first physiological data 60A may include ECAP data. In some examples, second physiological data 60B may include data different from first physiological data, such as data that is different from ECAP data when first physiological data 60A is ECAP data. In some examples, second physiological data 60B may include cardiac data, such as heart rate data.

In some examples, as shown in FIG. 3A, processing circuitry 50 may be configured to apply first physiological data 60A and first ML model 72 to the ML model hardware accelerator 20 to generate a first output 61A. In some examples, first output 61A may be stored on memory 56A. In some examples, as shown in FIG. 3B, processing circuitry 50 may be configured to apply second physiological data 60B and second ML model 74 to the ML model hardware accelerator 20 to generate a second output 61B. In some examples, second output 61B may be stored on memory 56A.

In some examples, processing circuitry 50 may send pointers 44 to at least one of memory 56A and ML model hardware accelerator 20 that indicates which data and ML model, such as first physiological data 60A and first ML model 72, to apply to ML model hardware accelerator 20, which may enable processing circuitry 50 to dynamically switch between ML models, such as dynamically switching between applying first ML model 72 and second ML model 74 to ML model hardware accelerator 20.

In accordance with techniques described herein, an IMD having two or more ML models that are stored on a shared memory and are configured to be executed via a shared ML model hardware accelerator on the IMD may improve power (e.g., battery power) efficiency and memory efficiency of an IMD running ML models. In some examples, since an IMD may have strict power and memory constraints, improving the power and/or memory efficiency, may enable greater usage of ML models in an IMD which may lead to improvements of the diagnostic and/or therapeutic capabilities of an IMD.

In some examples, processing circuitry 50 may be configured to determine a therapy to be provided, such as electrical stimulation, based on at least one of the first output 61A and the second output 61B. In some examples, processing circuitry 50 may be configured to determine an amplitude of the electrical stimulation to be provided, based on at least one of the first output 61A and the second output 61B. In some examples, in response to a determination of an amplitude of the electrical stimulation to be provided, processing circuitry 50 may be configured to cause at least one of a plurality of stimulation electrodes, such as electrodes of leads 150A and/or 150B, to generate the electrical stimulation at the determined amplitude.

In some examples, stimulation circuitry 57 may be configured to generate electrical stimulation therapy and processing circuitry 50 may be configured to adjust one or more parameters that define the electrical stimulation therapy 57 based on at least one of the first output 61A and the second output 61B. In some examples, processing circuitry 50 may be configured to adjust one or more parameters that define the electrical stimulation therapy based on at least one of the of the first output 61A and the second output 61B.

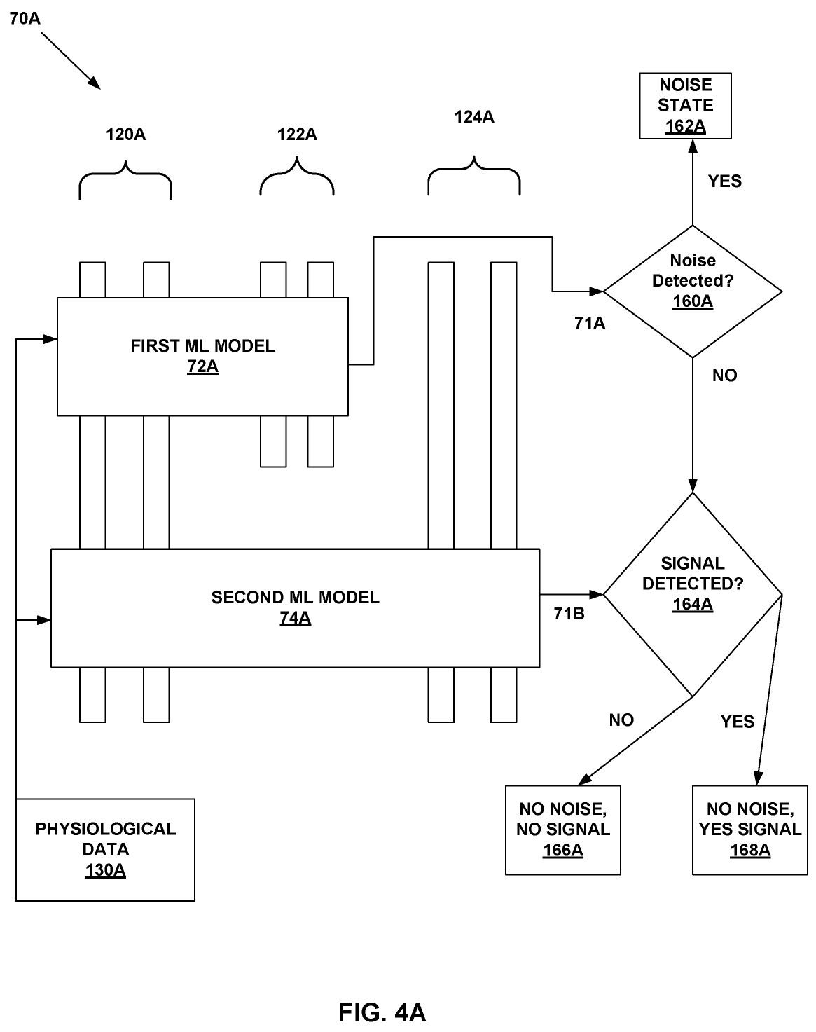

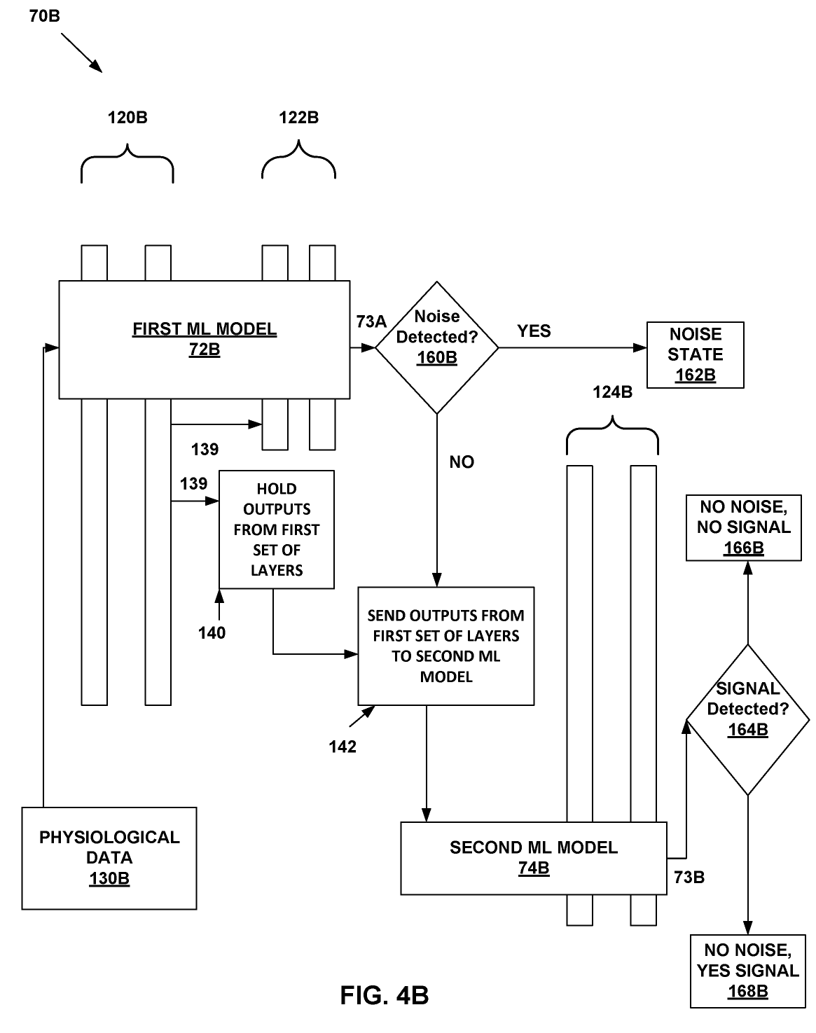

FIGS. 4A-4B are conceptual diagrams illustrating examples of system 70A, 70B configured to identify a physiological signal in sensed physiological data 130A, 130B to help determine a physiological parameter and/or health condition of a patient based on the identified physiological signal(s), such as by using a shared ML model hardware accelerator 20, according to the techniques of this disclosure.

FIG. 4A is an example technique of system 70A running a first ML model 72A and a second ML model 74A, that use a shared set of layers 120A (e.g., shared weights), in parallel to generate an output, such as determining whether a physiological signal is detected in sensed physiological data 130A. In some examples, system 70A determining a physiological signal is detected may correspond to the detected physiological signal including particular features of the signal to determine a physiological parameter and/or health condition of a patient based on features in the detected physiological signal(s).

In some examples, first ML model 72A and second ML model 74A may be stored on storage device 56 of IMD 10. In some examples, a first set of layers 120A, a second set of layers 122A, and a third set of layers 124A may be stored on storage device 56. In some examples, first ML model 72A may include the first set of layers 120A and the second set of layers 122A. In some examples, second ML model 74A may include the first set of layers 120A and the third set of layers 124A. In some examples, in system 70A the first ML model 72A and the second ML model 74A use a shared set of layers 120A while also then each using their own respective unique set of layers (e.g., second set of layers 122A for the first ML model 72A and third set of layers 124A for the second ML model 74A). In some examples, the layers of system 70A may be positioned in a ML model hardware accelerator 20, such as shown in FIGS. 3A-3B and the respective ML models 72A, 74A use particular sets of layers positioned in a shared ML model hardware accelerator to generate respective outputs.

In some examples, a layer may include interconnected nodes (or neurons) that perform specific computations. In some examples, a layer may comprises a linear transformation or non-linear transformation of an input into the layer. In some examples, a layer may be an input layer that may be configured to receive a sensed signal, a convolutional layer that may be configured to detect signal patters, a recurrent layer that may be configured to analyze temporal trends in the signal, or an output layer that is configured to determine an output, such as whether a physiological signal is detected in sensed physiological data, a stimulation parameter, or waveform classification. In some examples, where a particular layer is positioned with respect to other layers may indicate what type of layer the particular layer will be.

In some examples, an IMD may have limited memory size for an IMD to be small enough to be effectively implantable in a patient. The memory needs for running ML models are often high, which may be problematic for an IMD to run one or more ML models due to the limited memory size of an IMD. In accordance with some example techniques described herein, multiple ML models 72A, 74A using a shared set of layers, such the first set of layers 120A, instead of their own respective complete sets of layers, system 70A may reduce an amount of storage required by IMD 10 to store the sets of layers of the respective ML models 72A, 74A.

Physiological data 130 being sensed and collected by IMD 10 and processing circuitry 50 may apply physiological data 130 to the first ML model 72A and the second ML model 74A. In some examples, processing circuitry 50 may apply physiological data 130 to the first ML model 72A and the second ML model 74A simultaneously.

In some examples, first ML model 72A may be configured to detect whether noise in the sensed physiological data 130 satisfies a noise threshold. In some examples, second ML model 74A may be configured to detect whether a physiological signal, such as an ECAP signal, is detected in the physiological data 130. In some examples, output of second ML model 74A indicating a physiological signal, such as an ECAP signal, is detected may indicate the detected physiological signal includes sufficient particular features of the signal to determine a corresponding physiological parameter and/or health condition of a patient based on features in the detected physiological signal(s).

In some examples, processing circuitry 50 apply may physiological data 130A to the first ML model 72A to generate a first output 71A. In some examples, processing circuitry 50 may determine whether the first output 71A indicates whether noise has been detected (160A). For example, in response to determining noise is detected 162A in the physiological data (“YES” branch of block 160A), processing circuitry 50 may determine physiological data 130A includes noise and/or the IMD 10 is currently sensing in a noise state. In some examples, in response to a determination that noise is detected 162A in the physiological data 130A, processing circuitry 50 may output a signal indicating that noise is detected. In some examples, in response to a determination that noise is detected 162A in the physiological data 130, processing circuitry 50 may determine that a particular physiological signal, such as an ECAP signal, is not detected. In some examples, in response to determining noise is detected processing circuitry 50 may output an indication that IMD 10 is sensing in a noise state. In some examples, in response to determining noise is detected processing circuitry 50 may output an indication to not use physiological data 130A to determine features of the physiological data 130A.

In some examples, in response to determining noise is not detected in the physiological data (“NO” branch of block 160A), processing circuitry 50 may output an indication that no noise is detected 166A, 168A. In some examples, in response to determining noise is not detected in the physiological data (“NO” branch of block 160A), processing circuitry 50 may output an indication that the output of the second ML model 74A of whether a physiological signal was detected in physiological data to be output.

In some examples, processing circuitry 50 apply may physiological data 130 to the second ML model 74A to generate a second output 71B. In some examples, processing circuitry 50 may determine whether the second output 71B indicates whether a physiological signal, such as an ECAP signal, has been detected (164A). For example, in response to determining a physiological signal is detected 168A in the physiological data (“YES” branch of block 164A), processing circuitry 50 may determine physiological data 130A includes a sufficient signal, such as an ECAP signal, to be used to determine whether a particular health condition is present based on the signal. In some examples, in response to determining a physiological signal is detected 168A in the physiological data (“YES” branch of block 164A), processing circuitry 50 may perform feature extraction on the detected signal. In some examples, in response to determining a physiological signal is not detected 166A in the physiological data (“NO” branch of block 164A), processing circuitry 50 may determine physiological data 130A does not include a sufficient signal, such as an ECAP signal, to be used to determine whether a particular health condition is present based on the signal.

In some examples, processing circuitry 50 being configured to run the first ML model 72A and second ML model 74A in parallel may reduce latency in providing an indication of whether noise is detected in physiological data 130A or whether a physiological signal is detected in the sensed physiological data 130.

FIG. 4B is an example technique of system 70B running a first ML model 72B and a second ML model 74B, that use first layer set output 139 from a set of layers 120B, in series to determine whether a physiological signal is detected in sensed physiological data 130B. In some examples, system 70B determining a physiological signal is detected may include determining the physiological signal includes particular features to enable a determination of a physiological parameter and/or health condition of a patient based on the features in the physiological signal(s).

In some examples, first ML model 72B and second ML model 74B may be stored on storage device 56 of IMD 10. In some examples, a first set of layers 120B, a second set of layers 122B, and a third set of layers 124B may be stored on storage device 56. In some examples, first ML model 72B may include the first set of layers 120B and the second set of layers 122B. In some examples, second ML model 74B may include the third set of layers 124B. In some examples, second ML model 74B may include the first set of layers 120B and the third set of layers 124B. In some examples, in system 70B the first ML model 72B and the second ML model 74B both use first layer set output 139 from the first set of layers 120B to their own respective unique set of layers (e.g., second set of layers 122B for the first ML model 72B and third set of layers 124B for the second ML model 74B). In some examples, the layers of system 70B may be positioned in a ML model hardware accelerator 20, such as shown in FIGS. 3A-3B.

Physiological data 130B being sensed and collected by IMD 10 and processing circuitry 50 may apply physiological data 130B to the first ML model 72B. For example, processing circuitry 50 may apply physiological data 130B to the first set of layers 120B of the first ML model 72B.

In some examples, processing circuitry 50 may apply physiological data 130 to a first set of layers 120B to generate a first layer set output 139. For example, processing circuitry 50 apply may physiological data 130 to the first ML model 72B that includes first set of layers 120B and second set of layers 122B. In response to applying physiological data 130 to a first set of layers 120B to generate a first layer set output 139, processing circuitry 50 may the first layer set output 139 to the second set of layers 122B of the first ML model 72B to generate a first ML model output 73A. In some examples, processing circuitry 50 may additionally hold/store the first layer set output 139, such as in a memory or a temporary memory (140).

Processing circuitry 50 may determine whether first ML model output 73A satisfies a threshold, such as determining whether noise is detected based on whether the first ML model output 73A satisfies a threshold, such as a noise threshold (160B). For example, in response to determining noise is detected 162B in the physiological data (“YES” branch of block 160B), processing circuitry 50 may determine physiological data 130B includes noise and/or the IMD 10 is currently sensing in a noise state. In some examples, in response to a determination that noise is detected 162B in the physiological data 130B, processing circuitry 50 may output a signal indicating that noise is detected. In some examples, in response to a determination that noise is detected 162B in the physiological data 130, processing circuitry 50 may determine that a particular physiological signal, such as an ECAP signal, is not detected. In some examples, in response to determining noise is detected processing circuitry 50 may output an indication that IMD 10 is sensing in a noise state. In some examples, in response to determining noise is detected processing circuitry 50 may output an indication to not use physiological data 130B to determine features of physiological data 130B.

In some examples, in response to determining noise is not detected in the physiological data (“NO” branch of block 160B), processing circuitry 50 may output an indication to send first layer set output 139 to the second ML model 74B (142). In some examples, in response to a determination that the first ML model output 73A satisfies a threshold, such as a noise threshold (e.g., that noise is detected), processing circuitry 50 may apply the first layer set output 139 to the third set of layers 124B to generate a second ML model output 73B. In some examples, processing circuitry 50 may determine whether the second ML model output 73B indicates whether a physiological signal, such as an ECAP signal, has been detected (164B). In some examples, in response to determining a physiological signal is detected 168B in the physiological data (“YES” branch of block 164B), processing circuitry 50 may determine a physiological signal is detected in physiological data 130B and no noise is detected (168B). In some examples, the second ML model output 73B indicating a physiological signal is detected in physiological data 130B may indicate the detected physiological signal includes sufficient particular features of the signal to determine a corresponding physiological parameter and/or health condition of a patient based on features in the detected physiological signal(s). In some examples, in response to determining a physiological signal is not detected 166B in the physiological data (“NO” branch of block 164B), processing circuitry 50 may determine a physiological signal is not detected in physiological data 130B and no noise is detected (166B).

In some examples, an IMD may have limited memory size and battery power for an IMD to be small enough to be effectively implantable in a patient. The power needs for running ML models are often high, which may be problematic for an IMD to run one or more ML models due to the limited battery size of an IMD. In accordance with some example techniques described herein, multiple ML models 72B, 74B each using an output 139 of a set of layers, such the first set of layers 120B, and the second ML model 74B then applying the first layer set output 139 to the third set of layers 124B in response to the first ML model 74A determining noise is not present may save processing power and thus save battery usage. For example, by applying the first layer set output 139 to the first ML model 72B and holding/storing the first layer set output 139 to be applied to the second ML model 74B in response to determining noise is not detected in the physiological data 130B, the first set of layers 120B may only need to be run one time and the second ML model 74B may be executed when it is determined noise in the physiological data 130B does not exceed a threshold. In some examples, the techniques described herein may improve efficiency of the system 70B by not executing the second ML model 74B when determining noise in the physiological data 130B exceeds a threshold (e.g., the signal is too noisy) and by only executing the first set of layers 120B one time, instead of two times, when the second ML model 74B is executed. In some examples, system 70B may also reduce an amount of storage required by IMD 10 to store the sets of layers of the respective ML models 72B, 74B since an additional first set of layers 120B may not be required to be stored in storage device 56 of IMD 10.

In some examples, processing circuitry 50 being configured to run the first ML model 72B and second ML model 74B in series may reduce power usage by system 70B in providing an indication of whether noise is detected in physiological data 130B or whether a physiological signal is detected in the sensed physiological data 130B.

FIG. 5 is a block diagram illustrating an example configuration of components of external device 12. In the example of FIG. 4, external device 12 includes processing circuitry 80, communication circuitry 82, storage device 84, and user interface 86.

Processing circuitry 80 may include one or more processors that are configured to implement functionality and/or process instructions for execution within external device 12. For example, processing circuitry 80 may be capable of processing instructions stored in storage device 84. Processing circuitry 80 may include, for example, microprocessors, DSPs, ASICs, FPGAs, or equivalent discrete or integrated logic circuitry, or a combination of any of the foregoing devices or circuitry. Accordingly, processing circuitry 80 may include any suitable structure, whether in hardware, software, firmware, or any combination thereof, to perform the functions ascribed herein to processing circuitry 80.

Communication circuitry 82 may include any suitable hardware, firmware, software or any combination thereof for communicating with another device, such as IMD 10. Under the control of processing circuitry 80, communication circuitry 82 may receive downlink telemetry from, as well as send uplink telemetry to, IMD 10, or another device. Communication circuitry 82 may be configured to transmit or receive signals via inductive coupling, electromagnetic coupling, Near Field Communication (NFC), Radio Frequency (RF) communication, Bluetooth, WiFi, or other proprietary or non-proprietary wireless communication schemes. Communication circuitry 82 may also be configured to communicate with devices other than IMD 10 via any of a variety of forms of wired and/or wireless communication and/or network protocols.

Storage device 84 may be configured to store information within external device 12 during operation. Storage device 84 may include a computer-readable storage medium or computer-readable storage device. In some examples, storage device 84 includes one or more of a short-term memory or a long-term memory. Storage device 84 may include, for example, RAM, DRAM, SRAM, magnetic discs, optical discs, flash memories, or forms of EPROM or EEPROM. In some examples, storage device 84 is used to store data indicative of instructions for execution by processing circuitry 80. Storage device 84 may be used by software or applications running on external device 12 to temporarily store information during program execution.

Data exchanged between external device 12 and IMD 10 may include operational parameters. External device 12 may transmit data including computer readable instructions which, when implemented by IMD 10, may control IMD 10 to change one or more operational parameters and/or export collected data. For example, processing circuitry 80 may transmit an instruction to IMD 10 which requests IMD 10 to export collected data (e.g., digitized physiological signals) to external device 12. In turn, external device 12 may receive the collected data from IMD 10 and store the collected data in storage device 84. In some examples, storage device 84 may store a ML model.

A user, such as a clinician or patient 4, may interact with external device 12 through user interface 86. User interface 86 includes a display (not shown), such as a liquid crystal display (LCD) or a light emitting diode (LED) display or other type of screen, with which processing circuitry 80 may present information related to IMD 10, e.g., ECAPs, cardiac EGMs. In addition, user interface 86 may include an input mechanism to receive input from the user. The input mechanisms may include, for example, any one or more of buttons, a keypad (e.g., an alphanumeric keypad), a peripheral pointing device, a touch screen, or another input mechanism that allows the user to navigate through user interfaces presented by processing circuitry 80 of external device 12 and provide input. In other examples, user interface 86 also includes audio circuitry for providing audible notifications, instructions or other sounds to the user, receiving voice commands from the user, or both.

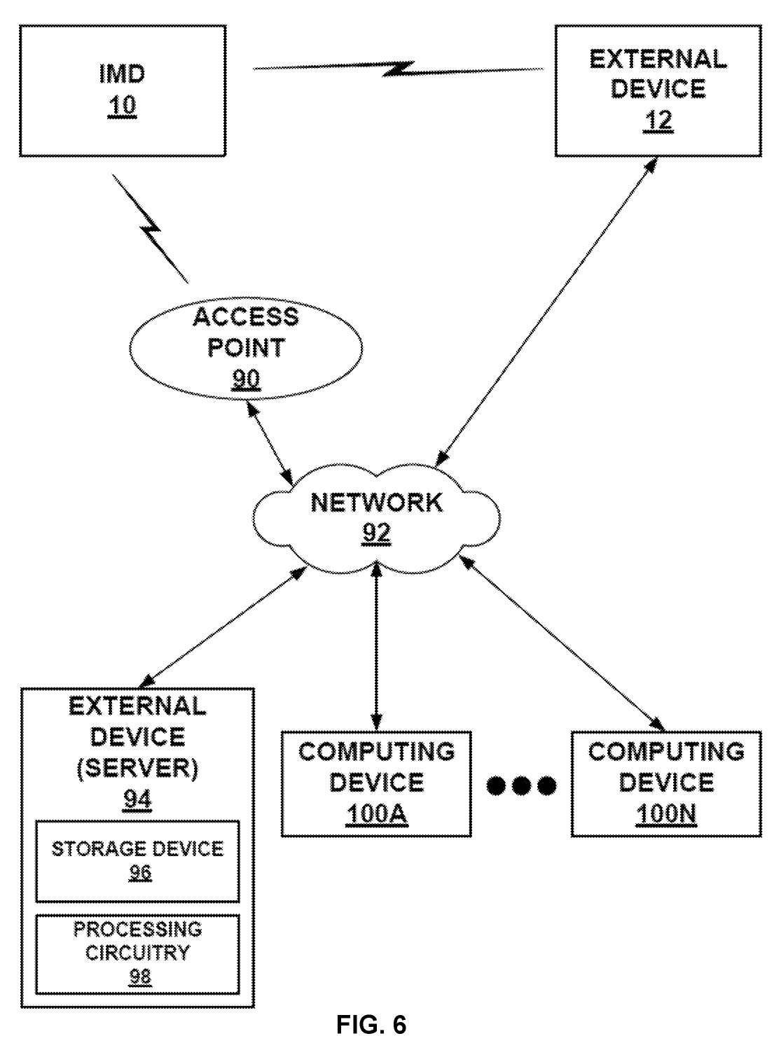

FIG. 6 is a block diagram illustrating an example system that includes an access point 90, a network 92, external computing devices, such as a server 94, and one or more other computing devices 100A–100N (collectively, “computing devices 100”), which may be coupled to IMD 10 and external device 12 via network 92, in accordance with one or more techniques described herein. In this example, IMD 10 may use communication circuitry 54 to communicate with external device 12 via a first wireless connection, and to communicate with an access point 90 via a second wireless connection. In the example of FIG. 6, access point 90, external device 12, server 94, and computing devices 100 are interconnected and may communicate with each other through network 92.

Access point 90 may include a device that connects to network 92 via any of a variety of connections, such as telephone dial-up, digital subscriber line (DSL), or cable modem connections. In other examples, access point 90 may be coupled to network 92 through different forms of connections, including wired or wireless connections. In some examples, access point 90 may be a user device, such as a tablet or smartphone, that may be co-located with the patient. IMD 10 may be configured to transmit data, such as physiological data, to access point 90. Access point 90 may then communicate the retrieved data to server 94 via network 92.

In some cases, server 94 may be configured to provide a secure storage site for data that has been collected from IMD 10 and/or external device 12. In some cases, server 94 may assemble data in web pages or other documents for viewing by trained professionals, such as clinicians, via computing devices 100. One or more aspects of the illustrated system of FIG. 6 may be implemented with general network technology and functionality, which may be similar to that provided by the Medtronic CareLink® Network.

In some examples, one or more of computing devices 100 may be a tablet or other smart device located with a clinician, by which the clinician may program, receive alerts from, and/or interrogate IMD 10. For example, the clinician may access data collected by IMD 10 through a computing device 100, such as when patient 4 is in in between clinician visits, to check on a status of a medical condition. In some examples, the clinician may enter instructions for a medical intervention for patient 4 into an application executed by computing device 100, such as based on a status of a patient condition determined by IMD 10, external device 12, server 94, or any combination thereof, or based on other patient data known to the clinician. Device 100 then may transmit the instructions for medical intervention to another of computing devices 100 located with patient 4 or a caregiver of patient 4. For example, such instructions for medical intervention may include an instruction to change a drug dosage, timing, or selection, to schedule a visit with the clinician, or to seek medical attention. In further examples, a computing device 100 may generate an alert to patient 4 based on a status of a medical condition of patient 4, which may enable patient 4 proactively to seek medical attention prior to receiving instructions for a medical intervention. In this manner, patient 4 may be empowered to take action, as needed, to address his or her medical status, which may help improve clinical outcomes for patient 4.

In the example illustrated by FIG. 6, server 94 includes a storage device 96, e.g., to store data retrieved from IMD 10, and processing circuitry 98. Although not illustrated in FIG. 6 computing devices 100 may similarly include a storage device and processing circuitry. Processing circuitry 98 may include one or more processors that are configured to implement functionality and/or process instructions for execution within server 94. For example, processing circuitry 98 may be capable of processing instructions stored in storage device 96. Processing circuitry 98 may include, for example, microprocessors, DSPs, ASICs, FPGAs, or equivalent discrete or integrated logic circuitry, or a combination of any of the foregoing devices or circuitry. Accordingly, processing circuitry 98 may include any suitable structure, whether in hardware, software, firmware, or any combination thereof, to perform the functions ascribed herein to processing circuitry 98.

Storage device 96 may include a computer-readable storage medium or computer-readable storage device. In some examples, storage device 96 includes one or more of a short-term memory or a long-term memory. Storage device 96 may include, for example, RAM, DRAM, SRAM, magnetic discs, optical discs, flash memories, or forms of EPROM or EEPROM. In some examples, storage device 96 is used to store data indicative of instructions for execution by processing circuitry 98.

Although the techniques for executing a plurality of ML models embedded on a medical device is described as being performed by processing circuitry 50 of IMD 10, such techniques may be performed, in whole or part, by processing circuitry of any one or more devices of system 2, such as processing circuitry 80 of external device 12, processing circuitry 98 of server 94, or processing circuitry of one or more computing devices 100.

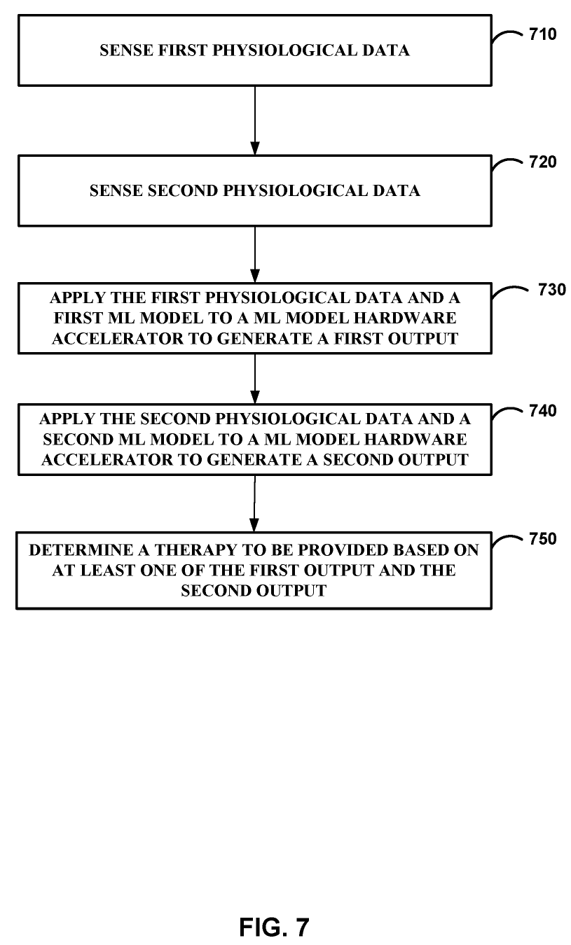

FIG. 7 is a flow diagram illustrating an example technique for IMD 10 to execute a first ML model 72 and a second ML model 74 using a shared ML model hardware accelerator 20. As indicated by FIG. 7, IMD 10 may be configured to sense first physiological data 60A of a patient 4 (710). In some examples, first physiological data 60A may be sensed by a first sensor of one or more sensors (e.g., sensors 62 and/or electrodes 16) of IMD 10. IMD 10 may be configured to sense second physiological data 60B of the patient 4 (720). In some examples, second physiological data 60B may be sensed by a second sensor of the one or more sensors (e.g., sensors 62 and/or electrodes 16) of IMD 10. In some examples, first physiological data 60A and second physiological data 60B may be different portions of a signal sensed by a same sensor of the one or more sensors (e.g., sensors 62 and/or electrodes 16) of IMD 10. Processing circuitry 50 may be configured to apply the first physiological data 60A and a first ML model 72 embedded on the IMD 10 to a ML model hardware accelerator 20 to generate a first output 61A (730). In some examples, the ML model hardware accelerator 20 is positioned in the IMD. In some examples, first physiological data 60A may include at least one on ECAP data, cardiac data, brain data, or other data. In some examples, second physiological data 60B may include at least one on ECAP data, cardiac data, brain data, or other data. In some examples, the second physiological data 60B is different than the first physiological data 60A. In some examples, the first sensor is different than the second sensor, such as being a different type of sensor configured to sense a different type of physiological data (e.g., first sensor being configured to sense ECAP data and the second being configured to sense cardiac data, such as heart rate).

Processing circuitry 50 may be configured to apply the second physiological data 60B and a second ML embedded on the IMD model to the ML model hardware accelerator to generate a second output 61B (740). Processing circuitry 50 can then be configured to determine a therapy to be provided to the patient based on at least one of the first output 61A and the second output 61B (750). The process of FIG. 7 may be performed in a continuous loop by continuing to apply sensed data to the ML modes programmed in order to provide continuous updates to the therapy. In some examples, this may be referred to as closed-loop therapy because it is being automatically controlled based on the sensed signals from the patient.

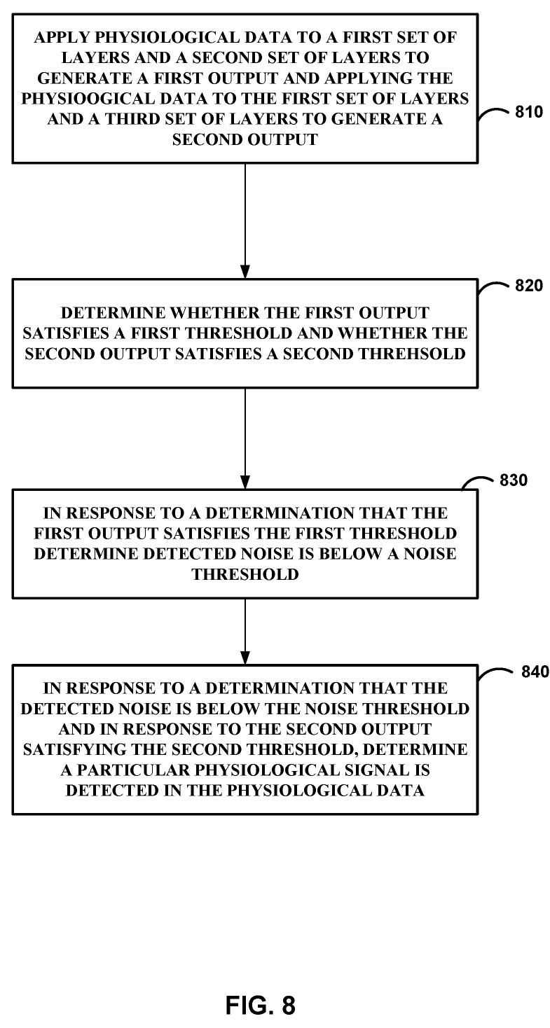

FIG. 8 is a flow diagram illustrating an example technique for running a first ML model 72A and a second ML model 74A, that use a shared set of layers 120A (e.g., shared weights), in parallel to generate an output, such as determining whether a physiological signal is detected in sensed physiological data 130A. In some examples, sensed physiological data 130A may include first physiological data sensed via a first sensor of the one or more sensors (e.g., sensors 62 and/or electrodes 16) of IMD 10 and second physiological data sensed via a second sensor of the one or more sensors (e.g., sensors 62 and/or electrodes 16) of IMD 10. In some examples, sensed physiological data 130A may include first physiological data and second physiological data that may be different portions of a signal sensed by a same sensor of the one or more sensors (e.g., sensors 62 and/or electrodes 16) of IMD 10. As shown in the example of FIG. 8, processing circuitry 50 may be configured to apply the physiological data 130A, such as physiological data sensed via sensors of IMD 10 (e.g., sensors 62 and/or electrodes 16), to a first set of layers 120A and a second set of layers 122A to generate a first output 71A and apply the physiological data 130A to the first set of layers 120A and the third set of layers 124A to generate a second output 71B (810). In some examples, storage device 56 may be configured to store a first ML model 72A comprising the first set of layers 120A and the second set of layers 122A. In some examples, storage device 56 may be configured to store a second ML model 74A comprising the first set of layers 120A and the third set of layers 124A. In some examples, the first set of layers 120A, the second set of layers 122A, and the third set of layers 124A are embedded on a ML model hardware accelerator 20 of IMD 10. In some examples, processing circuitry 50 may be configured to perform, in parallel, each of applying the physiological data 130A to the first set of layers 120A and the second set of layers 122A to generate the first output 71A and applying the physiological data 130A to the first set of layers 120A and the third set of layers 124A to generate the second output 71B.

Processing circuitry 50 may be configured to determine whether the first output 71A satisfies a first threshold and determine whether the second output 71B satisfies a second threshold (820). In response to a determination that the first output 71A satisfies the first threshold, processing circuitry 50 may determine the detected noise is below a noise threshold (e.g., an amount of noise in the physiological data 130A is low enough to perform analysis on the physiological data 130A) (830). In some examples, in response to a determination that the detected noise is below the noise threshold and in response to the second output 71B satisfying the second threshold, processing circuitry 50 may determine a particular physiological signal is detected in the physiological data 130A (84). In some examples, the particular physiological signal is an ECAP. In some examples, the particular physiological signal may be other signals, such as an ECG signal, EEG signal, respiration signal, optical signal, etc. The process of FIG. 8 may be performed in a continuous loop by continuing to apply sensed data to the ML modes programmed in order to provide continuous updates to the therapy. In some examples, this may be referred to as closed-loop therapy because it is being automatically controlled based on the sensed signals from the patient.

In some examples, stimulation circuitry 57 may be configured to generate electrical stimulation therapy and processing circuitry 50 may be configured to adjust one or more parameters that define the electrical stimulation therapy 57 based on at least one of the first output or the second output.

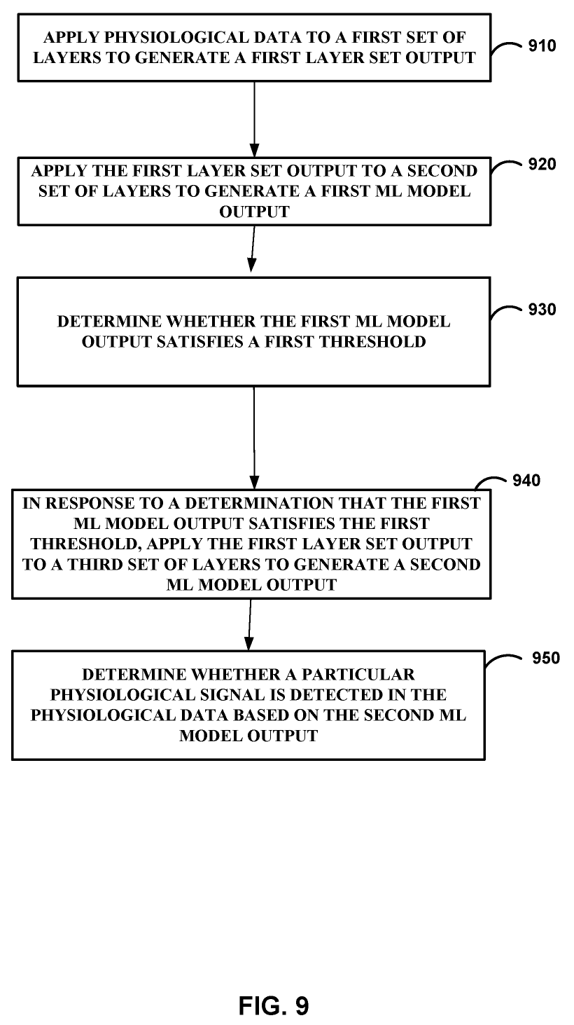

FIG. 9 is a flow diagram illustrating an example technique for running a first ML model 72B and a second ML model 74B in series using shared layer set outputs to generate an output, such as determining whether a physiological signal is detected in sensed physiological data 130B. In some examples, sensed physiological data 130B may include first physiological data sensed via a first sensor of the one or more sensors (e.g., sensors 62 and/or electrodes 16) of IMD 10 and second physiological data sensed via a second sensor of the one or more sensors (e.g., sensors 62 and/or electrodes 16) of IMD 10. In some examples, sensed physiological data 130B may include first physiological data and second physiological data that may be different portions of a signal sensed by a same sensor of the one or more sensors (e.g., sensors 62 and/or electrodes 16) of IMD 10. In some examples, a storage medium, such as storage device 56, may be configured to store first ML model 72B that comprises at least a first set of layers 120B and a second set of layers 122B. In some examples, a storage medium, such as storage device 56, may be configured to store second ML model 74B that comprises at least the first set of layers 120B and a third set of layers 124B. Processing circuitry 50 may be configured to apply the physiological data 130B, such as physiological data sensed via sensors of IMD 10 (e.g., sensors 62 and/or electrodes 16), to a first set of layers 120B to generate a first layer set output 139 (910). In some examples, processing circuitry 50 may be configured to store the first layer set output 139 in a memory of IMD 10, such as storage device 56, to be used later with respect to the second ML model 74B. Processing circuitry 50 may be configured to apply the first layer set output 139 to a second set of layers 122B to generate a first ML model output 73A (920). Processing circuitry 50 may be configured to determine whether the first ML model output 73A satisfies a first threshold (930).

In response to a determination that the first ML model output 73A satisfies the first threshold, apply the first layer set output 139, such as the first layer set output 139 being stored in a memory, to a third set of layers 124 to generate a second ML model output 73B (940). In some examples, storage device 56 may be configured to store first ML model 72B that comprises at least the first set of layers 120B and the second set of layers 122B. In some examples, storage device 56 may be configured to store second ML model 74B that comprises at least the first set of layers 120B and the third set of layers 124B. In some examples, the first set of layers 120B, the second set of layers 122B, and the third set of layers 124B are embedded on a ML model hardware accelerator 20 of the IMD 10. Processing circuitry 50 may be configured to determine whether a particular physiological signal is detected in the physiological data 130B based on the second ML model output 73B (950). In some examples, the particular physiological signal is an ECAP. In some examples, the particular physiological signal may be other signals, such as an ECG signal, EEG signal, respiration signal, optical signal, etc.

The process of FIG. 9 may be performed in a continuous loop by continuing to apply sensed data to the ML modes programmed in order to provide continuous determinations for newly sensed data. In this manner, this process may be a loop, or the system may restart the process for any new physiological data.

The following examples are illustrative of the techniques described herein.
Example 1: An implantable medical device includes a memory configured to store: first physiological data of a patient, second physiological data of the patient, a first machine learning (ML) model, and a second ML model; a ML model hardware accelerator; and processing circuitry configured to: apply the first physiological data and the first ML model to the ML model hardware accelerator to generate a first output; and apply at least one of the first physiological data or the second physiological data and the second ML model to the ML model hardware accelerator to generate a second output.
Example 2: The implantable medical device of example 1, further comprising a first sensor and a second sensor, wherein the first physiological data is sensed by the first sensor and the second physiological data is sensed by the second sensor.
Example 3: The implantable medical device of any of examples 1-2, wherein the first ML model is trained on data corresponding to the first physiological data.
Example 4: The implantable medical device of any of examples 1-3, wherein the second ML model is trained on data corresponding to the second physiological data.
Example 5: The implantable medical device of any of examples 1-4, wherein the first physiological data is representative of an evoked compound action potential (ECAP) of the patient.
Example 6: The implantable medical device of any of examples 1-5, wherein the processing circuitry is further configured to: control the implantable medical device to deliver a therapy based on at least one of the first output and the second output.
Example 7: The implantable medical device of example 6, wherein the therapy includes electrical stimulation, and wherein the processing circuitry is further configured to: adjust an amplitude of the electrical stimulation based on at least one of the first output and the second output.
Example 8: The implantable medical device of example 7, wherein the implantable medical device further comprises stimulation generation circuitry configured to couple to a plurality of stimulation electrodes, and wherein the processing circuitry is further configured to: control the stimulation generation circuitry at least one of the plurality of stimulation electrodes to generate the electrical stimulation at the determined amplitude.
Example 9: The implantable medical device of example 1, wherein the first physiological data and the second physiological data represent different portions of a signal sensed by a sensor.
Example 10: The implantable medical device of any of examples 1-9, wherein at least one of the first ML model and the second ML model are a neural network.
Example 11: The implantable medical device of any of examples 1-10, wherein the first ML model comprises a first set of layers and a second set of layers, and the second ML model comprises the first set of layers and a third set of layers, and wherein the first set of layers, the second set of layers, and the third set of layers are embedded on the ML model hardware accelerator.
Example 12: The implantable medical device of example 11, wherein the processing circuitry is further configured to: apply the first physiological data to the first set of layers and the second set of layers to generate the first output; and apply the second physiological data to the first set of layers and the third set of layers to generate the second output.
Example 13: The system of example 12, wherein the processing circuitry is configured to perform, in parallel, each of: apply the first physiological data to the first set of layers and the second set of layers to generate the first output; and apply the second physiological data to the first set of layers and the third set of layers to generate the second output.
Example 14: The implantable medical device of example 12, wherein the processing circuitry is further configured to: determine whether the first output satisfies a first threshold; determine whether the second output satisfies a second threshold; in response to the first output satisfying the first threshold, determine detected noise is below a noise threshold; and in response to a determination that the detected noise is below the noise threshold and in response to the second output satisfying the second threshold, determine whether a particular physiological signal is detected in at least one of the first physiological data or the second physiological data.
Example 15: The implantable medical device of example 11, wherein the processing circuitry is further configured to: apply the first physiological data to the first set of layers to generate a first layer set output; apply the first layer set output to the second set of layers to generate a first ML model output; determine whether the first ML model output satisfies a first threshold; and in response to the first ML model output satisfying the first threshold, apply the first layer set output to the third set of layers to generate a second ML model output.
Example 16: The system of example 15, wherein the processing circuitry is further configured to: store the first layer set output in the memory; and in response to the first ML model output satisfying the first threshold, apply the stored first layer set output to the third set of layers to generate the second ML model output.
Example 17: The system of any of examples 15-16, wherein the processing circuitry is further configured to: determine whether a particular physiological signal is detected in at least one of the first physiological data or the second physiological data based on the second ML model output.
Example 18: The system of any of examples 14 or 17, wherein the particular physiological signal is an evoked compound action potential (ECAP).
Example 19: A method includes sensing, by an implantable medical device (IMD), first physiological data of a patient; sensing, by the IMD, second physiological data of the patient; applying, by processing circuitry of the IMD, the first physiological data and a first machine learning (ML) model embedded on the IMD to a ML model hardware accelerator to generate a first output; and applying, by the processing circuitry, at least one of the first physiological data or the second physiological data and a second ML embedded on the IMD model to the ML model hardware accelerator to generate a second output.
Example 20: The method of example 19, wherein the first ML model comprises a first set of layers and a second set of layers, the second ML model comprises the first set of layers and a third set of layers, and the first set of layers, the second set of layers, and the third set of layers are embedded on the ML model hardware accelerator, the method further includes applying, by the processing circuitry, the first physiological data to a first set of layers and a second set of layers of the first ML model to generate the first output; applying, by the processing circuitry, the second physiological data to the first set of layers and a third set of layers of the second ML model to generate the second output; determining, by the processing circuitry, whether the first output satisfies a first threshold; and determining, by the processing circuitry, whether the second output satisfies a second threshold.
Example 21: The method of example 20, further includes in response to a determination that the first output satisfies the first threshold determining, by the processing circuitry, detected noise is below a noise threshold; and in response to determination that the detected noise is below a noise threshold and in response to the second output satisfying the second threshold, determining, by the processing circuitry, a particular physiological signal is detected in at least one of the first physiological data or the second physiological data.
Example 22: The method of example 19, wherein the first ML model comprises a first set of layers and a second set of layers, the second ML model comprises the first set of layers and a third set of layers, and the first set of layers, the second set of layers, and the third set of layers are embedded on the ML model hardware accelerator, the method further includes applying, by the processing circuitry, the first physiological data to the first set of layers to generate a first layer set output; applying, by the processing circuitry, the first layer set output to the second set of layers to generate the first output; determining, by the processing circuitry, whether the first output satisfies a first threshold; and in response to a determination that the first output satisfies the first threshold, applying, by the processing circuitry, the first layer set output to the third set of layers to generate the second output.
Example 23: The method of example 24 further includes determining, by the processing circuitry, whether a particular physiological signal is detected in at least one of the first physiological data or the second physiological data based on the second output.
Example 24: The method of any of examples 21 or 23, wherein the particular physiological signal is an evoked compound action potential (ECAP).
Example 25 The method of any of examples 22-23 further includes storing, by the processing circuitry, the first layer set output in a memory of the IMD; and in response to the first output satisfying the first threshold, applying the stored first layer set output to the third set of layers to generate the second output:
Example 26: A non-transitory computer-readable storage medium having stored thereon instructions that, when executed, cause one or more processors to perform any of the methods recited in any of examples 19-25.

The techniques described in this disclosure may be implemented, at least in part, in hardware, software, firmware, or any combination thereof. For example, various aspects of the techniques may be implemented within one or more microprocessors, DSPs, ASICs, FPGAs, or any other equivalent integrated or discrete logic QRS circuitry, as well as any combinations of such components, embodied in external devices, such as physician or patient programmers, stimulators, or other devices. The terms “processor” and “processing circuitry” may generally refer to any of the foregoing logic circuitry, alone or in combination with other logic circuitry, or any other equivalent circuitry, and alone or in combination with other digital or analog circuitry.

For aspects implemented in software, at least some of the functionality ascribed to the systems and devices described in this disclosure may be embodied as instructions on a computer-readable storage medium such as RAM, DRAM, SRAM, magnetic discs, optical discs, flash memories, or forms of EPROM or EEPROM. The instructions may be executed to support one or more aspects of the functionality described in this disclosure.

In addition, in some aspects, the functionality described herein may be provided within dedicated hardware and/or software modules. Depiction of different features as modules or units is intended to highlight different functional aspects and does not necessarily imply that such modules or units must be realized by separate hardware or software components. Rather, functionality associated with one or more modules or units may be performed by separate hardware or software components, or integrated within common or separate hardware or software components. Also, the techniques could be fully implemented in one or more circuits or logic elements. The techniques of this disclosure may be implemented in a wide variety of devices or apparatuses, including an IMD, an external programmer, a combination of an IMD and external programmer, an integrated circuit (IC) or a set of ICs, and/or discrete electrical circuitry, residing in an IMD and/or external programmer.

Various examples have been described. These and other examples are within the scope of the following claims.

What is claimed is:

1. An implantable medical device comprising:
a memory configured to store:
first physiological data of a patient,
second physiological data of the patient,
a first machine learning (ML) model, and
a second ML model;
a ML model hardware accelerator; and
processing circuitry configured to:
apply the first physiological data and the first ML model to the ML model hardware accelerator to generate a first output; and
apply at least one of the first physiological data or the second physiological data and the second ML model to the ML model hardware accelerator to generate a second output.

2. The implantable medical device of claim 1, further comprising a first sensor and a second sensor, wherein the first physiological data is sensed by the first sensor and the second physiological data is sensed by the second sensor.

3. The implantable medical device of claim 1, wherein the first ML model is trained on data corresponding to the first physiological data.

4. The implantable medical device of claim 3, wherein the second ML model is trained on data corresponding to the second physiological data.

5. The implantable medical device of claim 1, wherein the first physiological data is representative of an evoked compound action potential (ECAP) of the patient.

6. The implantable medical device of claim 1, wherein the processing circuitry is further configured to:
control the implantable medical device to deliver a therapy based on at least one of the first output and the second output.

7. The implantable medical device of claim 6, wherein the therapy includes electrical stimulation, and wherein the processing circuitry is further configured to:
adjust an amplitude of the electrical stimulation based on at least one of the first output and the second output.

8. The implantable medical device of claim 7, wherein the implantable medical device further comprises stimulation generation circuitry configured to couple to a plurality of stimulation electrodes, and wherein the processing circuitry is further configured to:
control the stimulation generation circuitry at least one of the plurality of stimulation electrodes to generate the electrical stimulation at the determined amplitude.

9. The implantable medical device of claim 1, wherein the first physiological data and the second physiological data represent different portions of a signal sensed by a sensor.

10. The implantable medical device of claim 1, wherein the first ML model comprises a first set of layers and a second set of layers, and the second ML model comprises the first set of layers and a third set of layers, and
wherein the first set of layers, the second set of layers, and the third set of layers are embedded on the ML model hardware accelerator.

11. The implantable medical device of claim 10,
wherein the processing circuitry is further configured to:
apply the first physiological data to the first set of layers and the second set of layers to generate the first output; and
apply the second physiological data to the first set of layers and the third set of layers to generate the second output.

12. The implantable medical device of claim 11, wherein the processing circuitry is configured to perform, in parallel, each of:
apply the first physiological data to the first set of layers and the second set of layers to generate the first output; and
apply the second physiological data to the first set of layers and the third set of layers to generate the second output.

13. The implantable medical device of claim 11, wherein the processing circuitry is further configured to:
determine whether the first output satisfies a first threshold;
determine whether the second output satisfies a second threshold;
in response to the first output satisfying the first threshold, determine detected noise is below a noise threshold; and
in response to a determination that the detected noise is below the noise threshold and in response to the second output satisfying the second threshold, determine whether a particular physiological signal is detected in at least one of the first physiological data or the second physiological data.

14. The implantable medical device of claim 10,
wherein the processing circuitry is further configured to:
apply the first physiological data to the first set of layers to generate a first layer set output;
apply the first layer set output to the second set of layers to generate a first ML model output;
determine whether the first ML model output satisfies a first threshold; and
in response to the first ML model output satisfying the first threshold, apply the first layer set output to the third set of layers to generate a second ML model output.

15. The implantable medical device of claim 14, wherein the processing circuitry is further configured to:
determine whether a particular physiological signal is detected in at least one of the first physiological data or the second physiological data based on the second ML model output.

16. A method comprising:
sensing, by an implantable medical device (IMD), first physiological data of a patient;
sensing, by the IMD, second physiological data of the patient;
applying, by processing circuitry of the IMD, the first physiological data and a first machine learning (ML) model embedded on the IMD to a ML model hardware accelerator to generate a first output; and
applying, by the processing circuitry, at least one of the first physiological data or the second physiological data and a second ML model embedded on the IMD to the ML model hardware accelerator to generate a second output.

17. The method of claim 16, wherein the first ML model comprises a first set of layers and a second set of layers, the second ML model comprises the first set of layers and a third set of layers, and the first set of layers, the second set of layers, and the third set of layers are embedded on the ML model hardware accelerator, the method further comprising:
applying, by the processing circuitry, the first physiological data to a first set of layers and a second set of layers of the first ML model to generate the first output;
applying, by the processing circuitry, the second physiological data to the first set of layers and a third set of layers of the second ML model to generate the second output;
determining, by the processing circuitry, whether the first output satisfies a first threshold; and
determining, by the processing circuitry, whether the second output satisfies a second threshold.

18. The method of claim 17, further comprising:
in response to a determination that the first output satisfies the first threshold determining, by the processing circuitry, detected noise is below a noise threshold; and
in response to determination that the detected noise is below a noise threshold and in response to the second output satisfying the second threshold, determining, by the processing circuitry, a particular physiological signal is detected in at least one of the first physiological data or the second physiological data.

19. The method of claim 16, wherein the first ML model comprises a first set of layers and a second set of layers, the second ML model comprises the first set of layers and a third set of layers, and the first set of layers, the second set of layers, and the third set of layers are embedded on the ML model hardware accelerator, the method further comprising:
applying, by the processing circuitry, the first physiological data to the first set of layers to generate a first layer set output;
applying, by the processing circuitry, the first layer set output to the second set of layers to generate the first output;
determining, by the processing circuitry, whether the first output satisfies a first threshold;
in response to a determination that the first output satisfies the first threshold, applying, by the processing circuitry, the first layer set output to the third set of layers to generate the second output; and
determining, by the processing circuitry, whether a particular physiological signal is detected in at least one of the first physiological data or the second physiological data based on the second output.

20. A non-transitory computer-readable storage medium having stored thereon instructions that, when executed, cause one or more processors of an implantable medical device (IMD) to:
control the IMD to sense first physiological data of a patient;
control the IMD to sense second physiological data of the patient;
apply the first physiological data and a first machine learning (ML) model embedded on the IMD to a ML model hardware accelerator to generate a first output; and
apply at least one of the first physiological data or the second physiological data and a second ML model embedded on the IMD to the ML model hardware accelerator to generate a second output.
